(19) United States
(12) Patent Application Publication
(10) Pub. No.: US 2008/0161810 A1
(43) Pub. Date: Jul. 3, 2008

(54) Guide and Cutter for Contouring Facet Joints and Methods of Use

(75) Inventor: Anthony J. Melkent, Memphis, TN (US)

Correspondence Address: COATS & BENNETT/MEDTRONICS, 1400 CRESCENT GREEN, SUITE 300, CARY, NC, 27518, US

(73) Assignee: Warsaw Orthopedic, Inc., Warsaw, IN (US)

(21) Appl. No.: 11/550,520

(22) Filed: Oct. 18, 2006

(51) Int. Cl. A61B 17/00

(52) U.S. Cl. 606/79

(57) ABSTRACT

A device and method for contouring articular processes of a facet joint includes a guide with a spacer extending outward in a longitudinal direction from a distal end of the guide. The spacer includes a width to space apart the articular processes in a widthwise direction. A cutting member that is offset the spacer in the widthwise direction includes a cutting edge oriented to contour one of the articular processes in the longitudinal direction. The cutting member may be fixed or moveable relative to the guide. If the cutting member is moveable relative to the guide, the guide may include a guide edge along which the cutting member moves to control the accuracy of the contouring. A second cutting member may be offset a second side of the spacer in the widthwise direction and include a second cutting edge oriented to contour the second articular process in the longitudinal direction.

### BACKGROUND

The human spine serves many functions. The vertebral members of the spinal column protect the spinal cord. The spinal column also supports other portions of the human body. Furthermore, moveable facet joints and resilient discs disposed between the vertebral members permit motion between individual vertebral members. Each vertebrae includes an anterior body and a posterior arch. The posterior arch includes two pedicles and two laminae that join together to form the spinous process. A transverse process is laterally positioned at the transition from the pedicles to the laminae. Both the spinous process and transverse process provide for attachment of fibrous tissue, including muscle. Two inferior articular processes extend downward from the junction of the laminae and the transverse process. Further, two superior articular processes extend upward from the junction. The articular processes of adjacent vertebrae form the facet joints. The inferior articular process of one vertebra articulates with the superior articular process of the vertebra below. The facet joints are referred to as gliding joints because the articular surfaces glide over each other.

Vertebral implants are often used in the surgical treatment of spinal disorders such as degenerative disc disease, disc herniations, curvature abnormalities, and trauma. Many different types of treatments are used. In some cases, spinal fusion is indicated to inhibit relative motion between vertebral bodies. Spinal fusion often involves the removal of the vertebral disc and insertion of an interbody implant to create a fused junction between a pair of vertebral bodies. Furthermore, the facet joints may be fused to complete the fusion between vertebral pairs. Facet fusion may be initiated by decorticating the opposing articulating surfaces and packing bone growth promoting substances into the space between the articular processes. The facet joints are generally small as compared to the intervertebral space. It may be difficult for the surgeon to determine the amount of contouring and shaping required for each of the articular processes. A trial-and-error routine is performed as the surgeon removes a first amount of material from one or both surfaces and determines whether the spacing is adequate for receiving a fusion device. Consequently, a certain amount of precision is desirable in preparing the articulating surfaces to receive a fusion implant and to prevent excessive trauma to the processes.

### SUMMARY

Illustrative embodiments disclosed herein are directed to devices and methods for contouring articular processes of a facet joint. The device may include a guide with a spacer extending outward in a longitudinal direction from a distal end of the guide. The spacer may include one or more prongs. The spacer includes a width to space apart the articular processes in a widthwise direction. A cutting member that is offset the spacer in the widthwise direction includes a cutting edge oriented to contour one of the articular processes in the longitudinal direction. The cutting member may be fixed or moveable relative to the guide. In either embodiment, a second cutting member may be offset a second side of the spacer in the widthwise direction and include a second cutting edge oriented to contour the second articular process in the longitudinal direction.

In embodiments where the cutting member is fixed with respect to the guides, the cutting edge may be proximally disposed relative to the guide. Contouring is performed by driving the guide into the facet joint. In embodiments where the cutting member is moveable relative to the guide, the guide may include a guide edge along which the cutting member moves to control the accuracy of the contouring. The cutting member may move laterally and/or in the longitudinal direction relative to the guide. Once the articular processes are prepared accordingly, an implant can be inserted between the processes to promote facet joint fusion.

### BRIEF DESCRIPTION OF THE DRAWINGS

FIG. 1 is a lateral view of a facet implant according to one embodiment shown relative to vertebral bodies;

FIG. 2 is an axial section view according to the section lines in FIG. 1;

FIG. 3 is an axial section view of a facet joint showing a cutting tool used to prepare the articular processes of the facet joint according to one embodiment;

FIG. 4 is a perspective detail view of a facet joint cutter according to one embodiment;

FIG. 5 is an axial section view of a facet joint showing a cutting tool preparing one articular process of the facet joint according to one embodiment;

FIG. 6 is an axial section view of a facet joint showing a cutting tool used to prepare the articular processes of the facet joint according to one embodiment;

FIG. 7 is a detail view of a facet joint cutter according to one embodiment;

FIG. 8 is an axial section view of a facet joint showing a cutting tool preparing one articular process of the facet joint according to one embodiment;

FIG. 9 is a detail view of a facet joint cutter according to one embodiment;

FIG. 10 is an axial section view of a prepared facet joint showing an implant insertable between the articular processes of the facet joint according to one embodiment;

FIG. 11 is a lateral view of an exemplary facet fusion implant according to one embodiment;

FIG. 12 is a top view of an exemplary facet fusion implant according to one embodiment;

FIG. 13 is an axial section view of a facet joint showing a guide used to prepare the articular processes of the facet joint according to one embodiment;

FIG. 14 is an axial section view of a facet joint showing a guide and cutting tool used to prepare the articular processes of the facet joint according to one embodiment;

FIG. 15 is a perspective view of a guide and cutting tool used to prepare the articular processes of a facet joint according to one embodiment;

FIG. 16 is a perspective view of a guide used to prepare the articular processes of a facet joint according to one embodiment;

FIG. 17 is a perspective view of a cutting tool used to prepare the articular processes of a facet joint according to one embodiment;

FIG. 18 is a perspective view of a cutting tool used to prepare the articular processes of a facet joint according to one embodiment;

FIGS. 19A and 19B are perspective views of a cutting tool used to prepare the articular processes of a facet joint according to one embodiment;

FIG. 20 is a perspective view of a guide used to prepare the articular processes of a facet joint according to one embodiment;

FIG. 21 is a perspective view of a guide used to prepare the articular processes of a facet joint according to one embodiment;

FIG. 22 is a perspective view of a guide used to prepare the articular processes of a facet joint according to one embodiment;

FIG. 23 is a perspective view of a guide used to prepare the articular processes of a facet joint according to one embodiment;

FIG. 24 is a perspective view of a guide used to prepare the articular processes of a facet joint according to one embodiment; and

FIG. 25 is a perspective view of a guide and cutting tool used to prepare the articular processes of a facet joint according to one embodiment.

### DETAILED DESCRIPTION

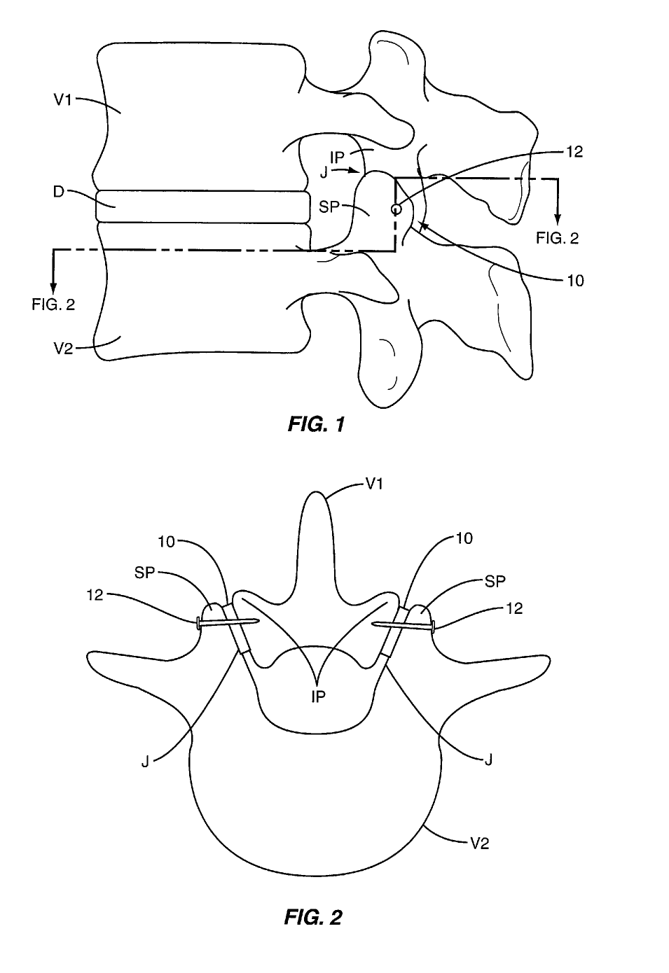

The various embodiments disclosed herein relate to methods and devices used in the preparation of a facet joint to promote fusion of the facet in spinal fusion surgery. FIG. 1 illustrates one embodiment of an implant 10 installed according to this approach. Specifically, FIG. 1 shows a lateral view of two vertebrae V1, V2 and an intervertebral disc D disposed therebetween. During fusion surgery, some or the entire disc D is removed and may be replaced with an implant or graft that ultimately fuses to the vertebrae V1, V2. In addition, a surgeon may elect to fuse the facet joints J that are formed between the inferior articular process IP of the superior vertebra V1 and the superior articular process SP of the inferior vertebra V2. To that end, the implant 10 may be inserted between the articular processes IP, SP as illustrated and disclosed herein. The section view shown in FIG. 2 is depicted according to the section line labeled II-II in FIG. 1. The exemplary implant 10 illustrated in FIG. 1 is also presented in FIG. 2. In one or more embodiments, the implant 10 may be pinned, screwed, or otherwise secured to the articular processes IP, SP using a fastener 12. The fastener 12 may be implemented using a pin, a nail, a screw, a staple, a band, or other feature that secures the implant 10 to the facet joint J until fusion occurs. Other embodiments disclosed herein may be implemented without a fastener 12.

The implant 10 and fastener 12 may be constructed of biocompatible materials, including metals, such as titanium or stainless steel, non-metals, such as PEEK or UHMWPE. The implant 10 and fastener 12 may be constructed of a graft material, which is interpreted herein to include implants constructed from natural or synthetic bone materials including, but not limited to autograft, allograft, xenograft, or calcium phosphate. In embodiments where the implant 10 is constructed from synthetic or manufactured materials, the implant 10 may be coated or textured to improve the likelihood of bony ingrowth into the implant. Similarly, the implant may be impregnated, packed, or filled with bone growth promoting substances such as Bone Morphogenetic Protein (BMP), demineralized bone matrix (DBM), allograft, autograft, xenograft, or other osteoinductive growth factors. For example, the implant 10 may include a porous structure with open portions of the implant 10 packed with the bone growth promoting substance. In certain implementations, the implant 10 may osseointegrate or become part of the fusion mass at the facet joint J to increase the size and stability of the fusion mass. In one embodiment, the fastener 12 may be constructed from a bioabsorbable material that begins to dissolve after the implant 10 has begun to fuse to the facet joint J.

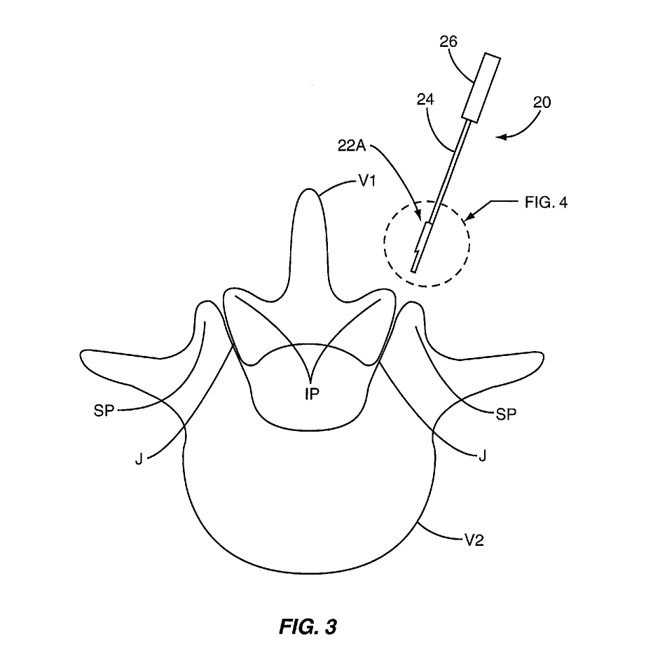

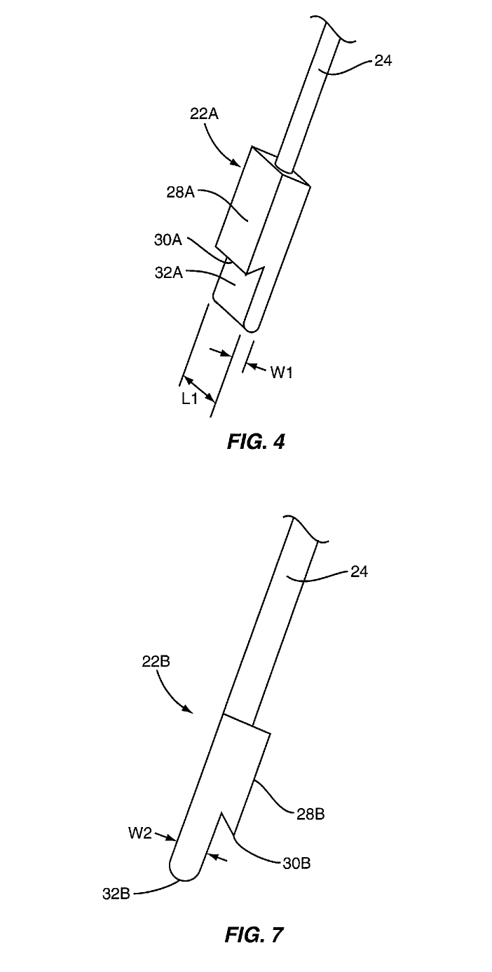

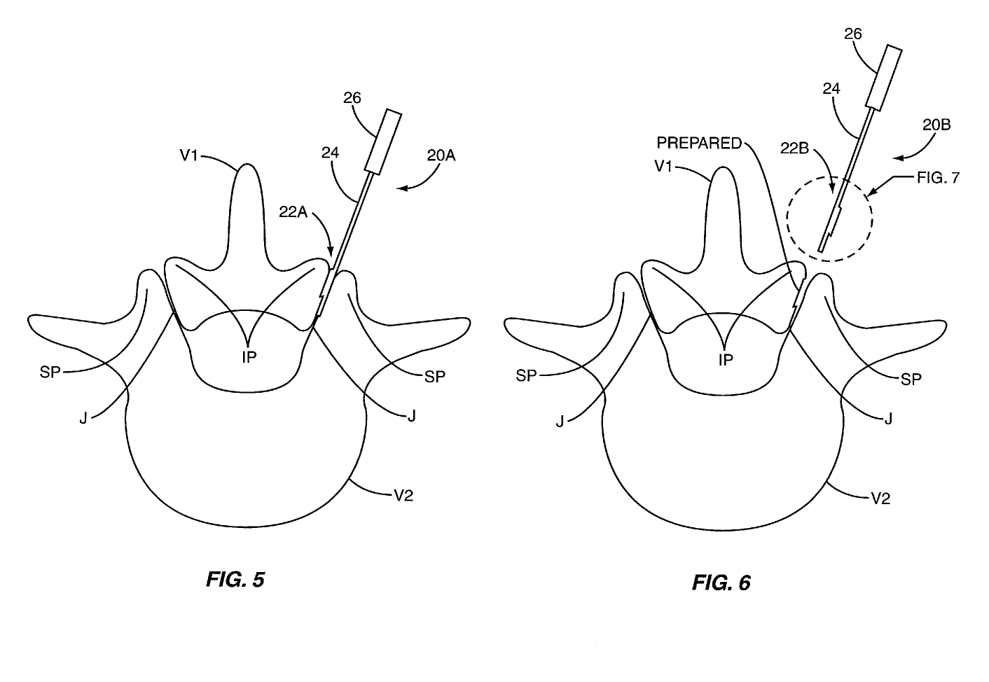

The facet joint J may be prepared in advance of receiving the implant 10 by decorticating the articulating surfaces of the joint J. FIG. 3 illustrates one embodiment of a cutting tool 20A adapted for this purpose. The cutting tool 20A includes a handle 26, elongated shaft 24, and cutter 22A disposed at the distal end of the tool 20A. FIG. 4 illustrates a detailed perspective view of the cutter 22A according to the drawing callout in FIG. 3. The illustrated embodiment includes a spacer guide 32A at the distal end of the cutting tool 20A and a blade 28A proximally disposed on one side of the guide 32A. The blade 28A includes a sharpened leading edge 30A that is configured to remove cartilage and/or bone from one of the articular processes that form the facet joint J. The guide 32A includes a width W1 that enters the facet joint J (as shown in FIG. 5) and keeps the blade 28A parallel to the joint J. The guide 32A and blade 28A include a length L1 in the transverse direction that is at least as wide as an implant 10 that is inserted into the joint J. The cutting tool 20A may be driven into the joint J through impact with the handle 26. Other conventionally known techniques, including manual, electric, and pneumatic drivers, may be used to drive the cutter 22A through the facet joint J thereby removing bone and cartilage from one of the two processes. Notably, FIG. 5 shows the inferior process IP of vertebra V1 being prepared in this manner.

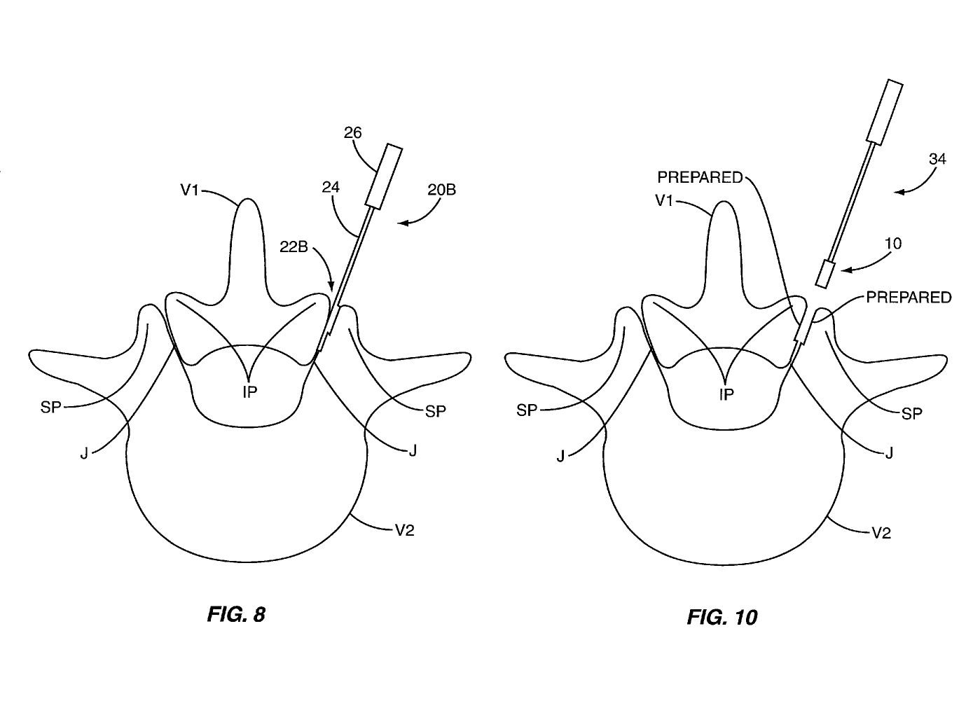

After one of the two articular processes IP, SP is prepared, a second cutting tool 20B may be used to prepare the other of the two processes IP, SP. In the embodiment shown in FIG. 6, the second cutting tool 20B is used to remove cartilage and bone from the superior process SP of vertebra V2. The second cutting tool 20B is similar in form to the first cutting tool 20A in that it includes a handle 26, elongated shaft 24, and cutter 22B disposed at the distal end of the tool 20B. FIG. 7 illustrates a detailed view of the cutter 22B. The illustrated embodiment includes a spacer guide 32B at the distal end of the cutting tool 20B and a blade 28B proximally disposed on one side of the guide 32B. The blade 28B includes a sharpened leading edge 30B that is configured to remove cartilage and/or bone from one of the articular processes that form the facet joint J. The guide 32B includes a width W2 that is greater than the width W1 of the first guide 32A. The additional width accounts for material that is removed by the first cutting tool 20A. The second guide 32B enters the facet joint J (as shown in FIG. 8), moving along the previously prepared surface and keeps the blade 28B parallel to the joint J. As before, the cutting tool 20B may be driven into the joint J through impact with the handle 26, other conventionally known driving techniques.

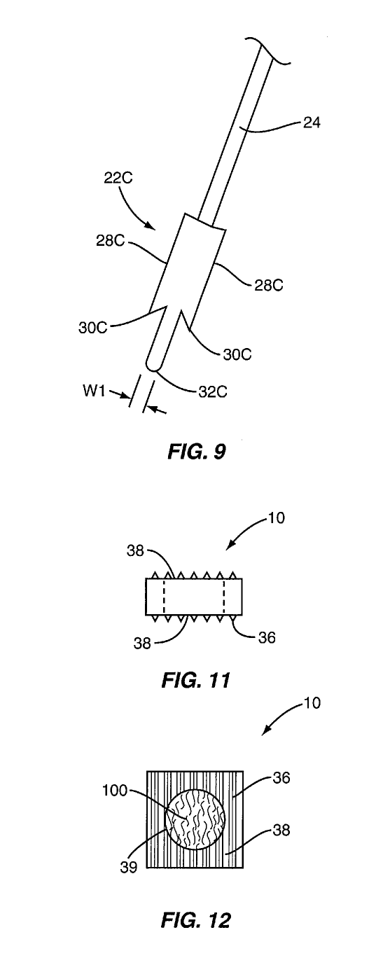

FIG. 9 shows an embodiment of a cutter 22C including blades 28C that are proximally disposed on opposite sides of the spacer guide 32C. The guide 32C includes a width W1 that is substantially similar to that of the guide 32A on the first cutting tool 20A. In the embodiment shown, the blades 28C are positioned so that the leading cutting edge 30C of each blade is disposed at substantially the same longitudinal position. In an unillustrated embodiment, the blades 28C may be staggered slightly so that each initially engages bone at different times. In either case, the illustrated cutter 22C may permit preparation of both articulating faces of the facet joint J using a single cutting tool.

Once the opposing surfaces of the joint J are prepared as shown in FIG. 10, the implant 10 may be placed into the facet joint J using an insertion tool 34. As suggested above, the implant 10 is intended to fuse with the previously articulating processes IP, SP, thereby fusing the vertebrae V1, V2 at the facet joint J. To that end, the implant 10 may include various features to promote fusion. FIGS. 11 and 12 illustrate one exemplary embodiment of an implant 10 that may be used for facet fusion. The implant 10 includes surface features 36 to promote bone growth and adhesion at the interface between the implant 10 and articulating processes IP, SP. Examples of features used for this purpose include, for example, teeth, scales, keels, knurls, and roughened surfaces. Some of these features 36 may be applied through post-processing techniques such as blasting, chemical etching, and coating, such as with hydroxyapatite. The bone interface surfaces 38 may also include growth-promoting additives such as bone morphogenetic proteins. Alternatively, pores, cavities, or other recesses into which bone may grow may be incorporated via a molding process. Other types of coatings or surface preparation may be used to improve bone growth into or through the bone-contact surfaces 38. FIG. 12 further shows that the implant 10 may include one or more apertures 39 that may be packed with bone growth promoting material 100 in an attempt to promote new bone growth that will ultimately fuse the facet joint J. Some non-limiting examples of bone growth promoting substances that may be inserted into the aperture 39 include Bone Morphogenetic Protein (BMP), demineralized bone matrix (DBM), allograft, autograft, xenograft, or other osteoinductive growth factors to facilitate fusion of the facet joint J. Further, the shape of the exemplary implant 10 is substantially rectangular. However, the implant 10 may assume other shapes, including for example, round, square, oval, polygonal, or other shapes that would occur to one skilled in the art. During the insertion procedure, the implant 10 may be adhered to the articular processes IP, SP with a biocompatible adhesive. Suitable adhesives may include protein derived, aldehyde based adhesive materials, albumin/glutaraldehyde materials, and cyanoacrylate-based materials.

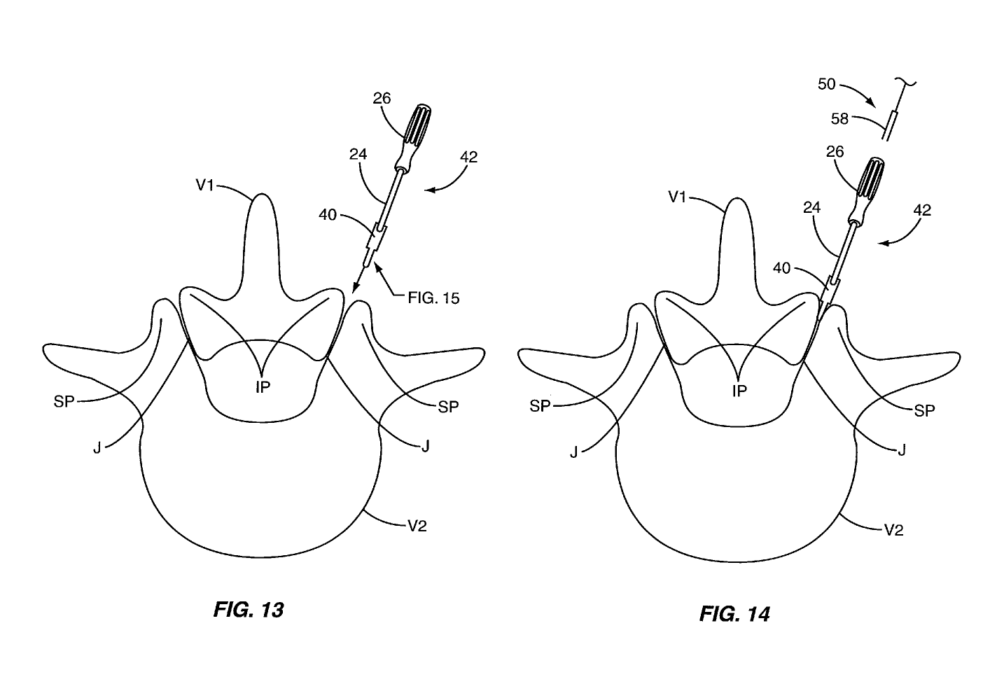

In embodiments described above, the guides 32A-C and blades 28A-C formed a part of the same cutter 22A-C. In other embodiments, the guide and blade may be separated into different bodies. FIGS. 13 and 14 illustrate one such embodiment where a hand-held guide body 40 is inserted into the facet joint J in a first step. Subsequently, a cutting tool 50, including one or more blades 58, is inserted through the guide body 40 to prepare the articulating surfaces of the processes IP, SP. As discussed in greater detail below, motion of the cutting tool 50 is constrained by the guide body 40 to control the joint J preparation. In the embodiment shown, the guide body 40 is formed at a distal end of a guide tool 42 that includes a handle 26 and elongated arm 24 that allow a surgeon to accurately position the guide body 40 relative to the processes IP, SP.

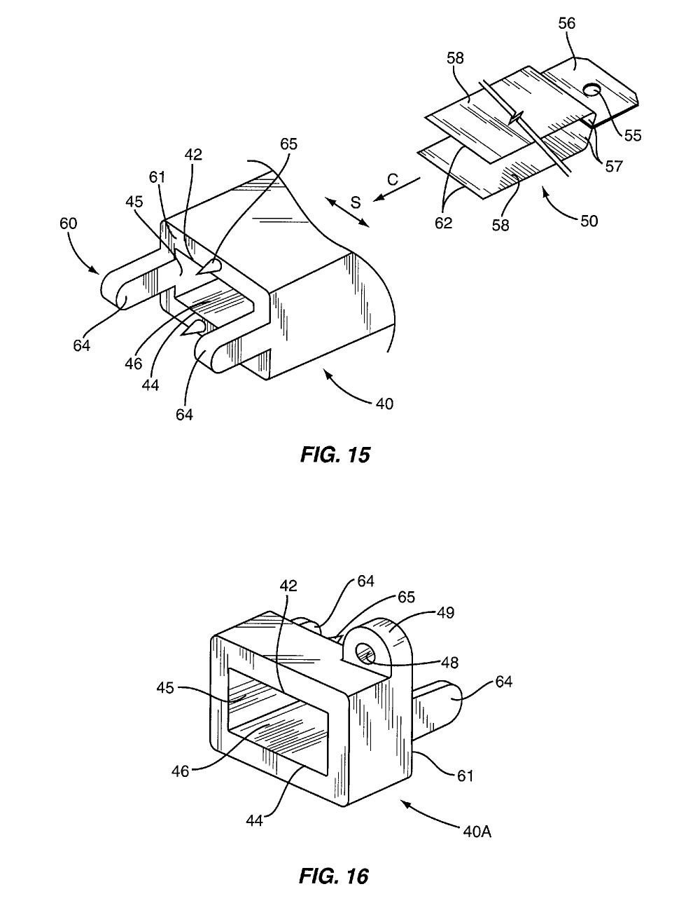

FIG. 15 depicts a perspective view of the exemplary guide body 40 viewed in the general direction of the arrow labeled XV in FIG. 13. The guide body 40 is sized to be mounted between articular processes of a facet joint J and has a pair of edges 42, 44 that are spaced for receiving blades 58 of the cutting tool 50. The pair of edges 42, 44 are spaced a distance apart for the first edge 42 to align with a first articular process, and the second edge 44 to align with a second articular process. The cutting tool 50 in the illustrated embodiment includes a pair of blades 58, with respective cutting edges 62. The blades 58 are sized to fit between the pair of edges 42, 44 respectively. The cutting edges 62 are positioned at a distal end of the blades 58 with a mount 56 at the proximal end. The mount may include apertures 55 for attachment to a driver. Using the cutting tool 50 and guide body 40 comprises inserting the guide body 40 adjacent a facet joint J and inserting the blades 58 through the pair of edges 42, 44. The edges 42, 44 are sized for the blades 58 to move within and contour the members. The guide body 40 and cutting tool 50 are constructed of rigid materials, such as stainless steel, though other materials, including other metals or non-metals may be used.

The guide body 40 is sized such that the first and second edges 42, 44 extend through the guide body 40 and position the cutting tool 50 at the proper placement relative to the facet joint J and support the blades 58 during the contouring procedure. The guide body 40 may also act as a spacer to position articular processes IP, SP of the facet joint J an appropriate distance apart for performing the contouring process. Facet joint J spacing may be achieved with a spacer 60, including one or more prongs 64 as described in greater detail below. In one embodiment, guide body 40 is constructed of a unitary member. In another embodiment, guide body 40 is an assembled part comprising two or more different sections.

In the embodiment illustrated in FIGS. 14 and 15, the first and second edges 42, 44 are part of an interior aperture 46 that is sized to receive the cutting tool 50. Lateral walls 45 define the overall width of the aperture 46 and edges 42, 44. In some embodiments, the aperture 46 and lateral walls 45 extend between the first and second edges 42, 44. The aperture 46 is sized for the surgeon to visually observe the contouring process. The aperture 46 further allows for access to the facet joint J for irrigation and bone removal during the contouring process. Aperture 46 may include a variety of sizes and shapes depending upon the application. Thus, in addition to the rectangular shape shown, the aperture may include, for example, oval, hourglass, or I-beam shapes. For example, FIG. 25 illustrates a guide body 40 with the aforementioned spacer 60 and an inner aperture 46 that is rounded. A corresponding cutting tool 50 includes a rounded shape with a circular leading edge 62. The leading edge 62 may be serrated. The cutting tool 50 may be impacted in the direction of arrow C. Alternatively, the cutting tool 50 may be used to contour the articular processes IP, SP by rotating the cutting tool 50 in the direction of arrow R. Other types of rotary cutting tools known in the art, including broaches, drilling bits, reamers, and other fluted or non-fluted boring tools, may be used in conjunction with the guide body in FIG. 25.

Referring again to FIG. 15, the edges 42, 44 include a width greater than the blades 58 to provide room for the cutting tool 50 to pass during the contouring process. In one embodiment, the edges 42, 44 may be sized for the blades 58 to pass through the guide body 40 with motion substantially constrained in the direction of arrow C. In one embodiment, the edges 42, 44 may be sized for the blades 58 to pass through in the direction of arrow C as well as reciprocate back and forth in the direction of arrow S. The edges 42, 44 may be sharpened to remove bone material from the articular processes IP, SP as the cutting tool 50 is driven or impacted in the direction of arrow C. Further, cutting tool 50 movement may be oscillating, reciprocating, vibratory, and other known manners. The first and second edges 42, 44 may include a variety of shapes depending upon the specific application. In the embodiment illustrated, the first and second edges 42, 44 are straight and parallel to contour parallel surfaces of the adjacent articular processes IP, SP. However, depending upon the application, other embodiments may be included such as curved edges and jagged edges. In one embodiment, the blades 58 are shaped to conform to the shape of the edges 42, 44.

In the illustrated embodiment, a spacer 60 extends from a distal face 61 of the guide body 40 for spacing the articular processes IP, SP. Spacer 60 may include a variety of shapes to fit between the articular processes IP, SP and space them a predetermined distance apart. In one embodiment, spacer 60 includes two prongs 64 that extend outward from the guide body 40. Spacer 60 is spaced between the first and second edges 42, 44 to not interfere with access to the articular processes IP, SP during the contouring. Worded in another manner, the distance between the first and second edges 42, 44 is about equal to or greater than the width of the spacer 60. In one embodiment, the distance between the first and second edges 42, 44 is about the same as the width of the spacer 60 such that the reference edges 42, 44 align with the edges of the articular processes IP, SP to contour only a small amount. In another embodiment, the distance between the first and second edges 42, 44 is greater than the width of the spacer 60 such that reference edges 42, 44 align further on the vertebral members to contour a larger amount.

In the embodiment shown in FIGS. 14 and 15, the guide body 40 is attached to a handle 26 for positioning within the facet joint J. In other embodiments, such as the guide body 40A shown in FIG. 16, apertures 48 may be positioned for attaching the guide body 40A to the articular processes IP, SP. Note that in each of the various embodiments disclosed herein, the guide body 40 may include one or both of the mounting apertures 18 and the elongated arm 24 and handle 26. Apertures 48 may be spaced at a variety of locations about the guide body 40A. The apertures 48 may be formed within a tab 49 that extends from the guide body 40A. In one embodiment, the apertures 48 are located on opposite sides of the guide body 40A such that a first aperture 48 is aligned with a first articular process IP, SP and a second aperture (not visible in FIG. 16) is aligned with a second articular process IP, SP. Each aperture 48 is sized to receive a fastener (not shown) such as a bone screw, nail, or staple for at least temporary connection to the articular processes IP, SP. Further, in one or more embodiments, the distal face 61 of the guide body 40, 40A may include surface features 65 such as teeth, spikes, scales, knurls, or roughened surfaces to penetrate the bone adjacent to the facet joint J to further secure the guide relative to the facet joint J while cutting tool 50 contours the articular processes IP, SP.

In one embodiment, cutting tool 50 comprises first and second blades 58 extending a distance apart by a span 57. A mount 56 is positioned opposite the blades 58 for attachment to a drive source. In one embodiment, blades 58 are the same length such that cutting edges 62 at the distal end are aligned and contour the articular processes IP, SP to the same depth. As suggested above, the blades 58 are spaced a distance apart to align respectively with the first and second edges 42, 44. In one embodiment, blades 58 are parallel and span 57 is substantially perpendicular. The height of the cutting tool 50 is the distance extending between the edges 62 and the span 57.

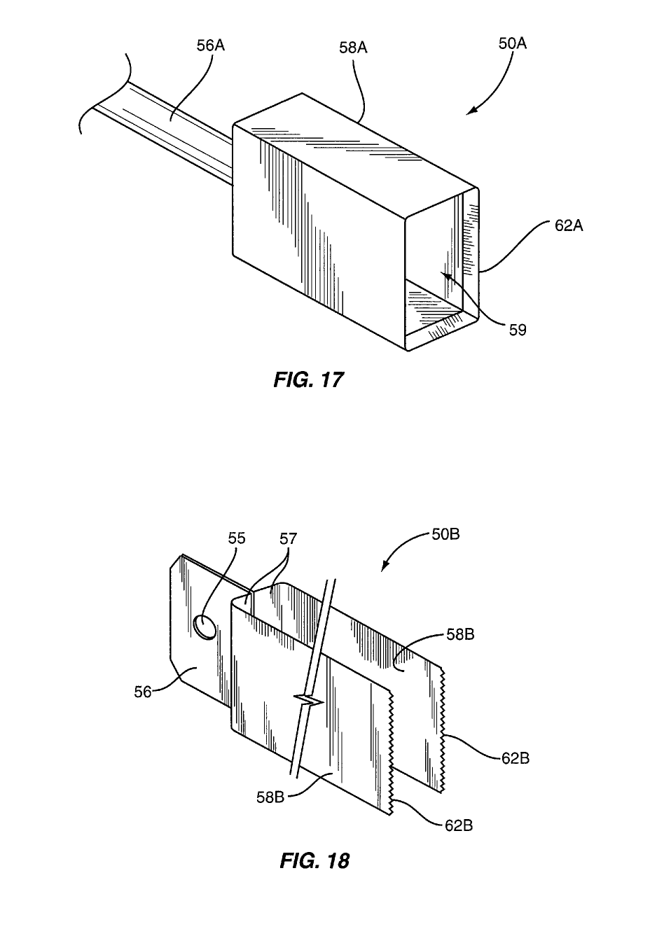

The cutting tool 50 may assume a variety of shapes and configurations. In the embodiment shown in FIG. 15, the cutting edges 62 are sharpened and substantially rigid to effectively chisel cartilage and bone matter from the articular processes IP, SP. In an embodiment illustrated in FIG. 17, the cutting tool 50A includes a boxed configuration in which a rectangular hollow body 58A terminates at a rectangular cutting edge 62A. The interior 59 of the body 58A is substantially empty or may include structural reinforcement (not shown) to strengthen the cutting tool 50A. The cutting tool 50A further includes an elongated mount 56A for attachment to a driver. In one embodiment shown in FIG. 18, the cutting tool 50B includes blades 58B with cutting edges 62A that are serrated and include a plurality of individual teeth that contact the articular processes IP, SP. Other types of cutting edges 62A with different teeth sizes and orientations are known and may be used.

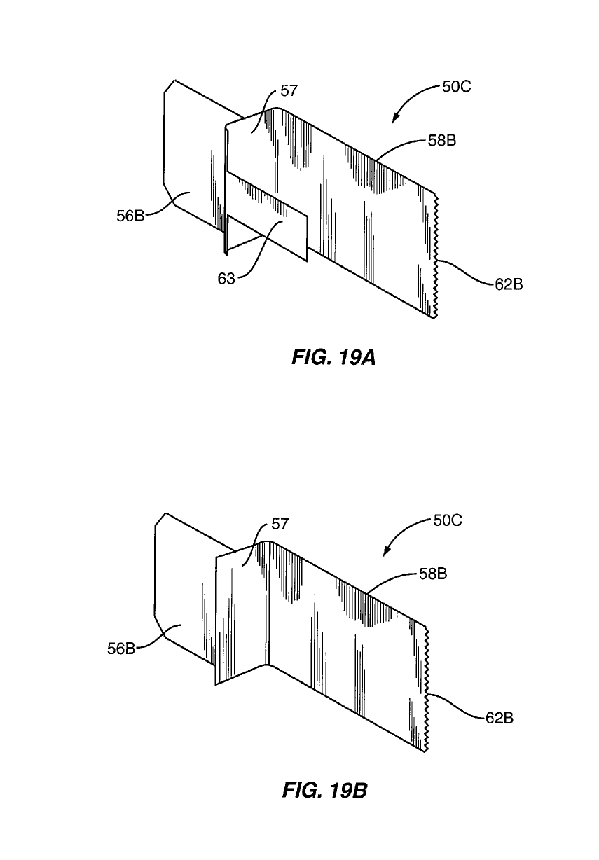

FIG. 19A illustrates another embodiment of a cutting tool 50C that comprises a single blade 58B with an associated cutting edge 62B. The cutting tool 50C may include a guide arm 63 serving as a guide to contact a first or second edge 42, 44 to position the single blade 58B. Guide arm 63 does not include a cutting edge 62. The guide arm 63 is sized to contact one of the reference edges 42, 44 and may include a number of different shapes and sizes. The guide arm 63 may include a variety of lengths, provided it is not of such a length to contact the articular processes IP, SP and interfere with the contouring process. In the illustrated embodiment, the span 57 may be narrower or wider than the distance between the reference edges 42, 44. FIG. 19B illustrates a similar embodiment of a cutting tool 50C that comprises a single blade 58B with an associated cutting edge 62B, but without a guide arm 63. In one unillustrated embodiment, the cutting device 50 can include a single or double wheel cutter that passes through the guide body 40 to contour the articular processes IP, SP.

In each of the above cutting tool embodiments, the mounts 56, 56A, 56B provide for attaching the cutting tool 50 to a driving device. Other mounts may include a variety of shapes and sizes suitable for a particular driving device and/or application. The mounts 56, 56A, 56B may be attached to a handle that is impacted to drive the cutting tools 50, 50A-C into the articular processes IP, SP. In certain embodiments, apertures 55 may be positioned for attaching the cutting tools 50, 50B to a driving device. The cutting tools 50, 50A-C may be attached to a power device, such as a reciprocating saw (not shown). A variety of different power sources may drive the cutting tools 50, 50A-C. Embodiments include a rechargeable battery, pneumatic mechanism, and any standard electrical source, such as 110 volt, 60 cycle power sources, with or without a transformer to reduce the voltage as necessary. In one embodiment, the cutting tool 50, 50A-C is oscillated back and forth in a direction parallel with or aligned with the first and second edges 42, 44. In another embodiment, cutting tool 50, 50A-C is oscillated in an in-and-out direction substantially perpendicular to the first and second edges 42, 44.

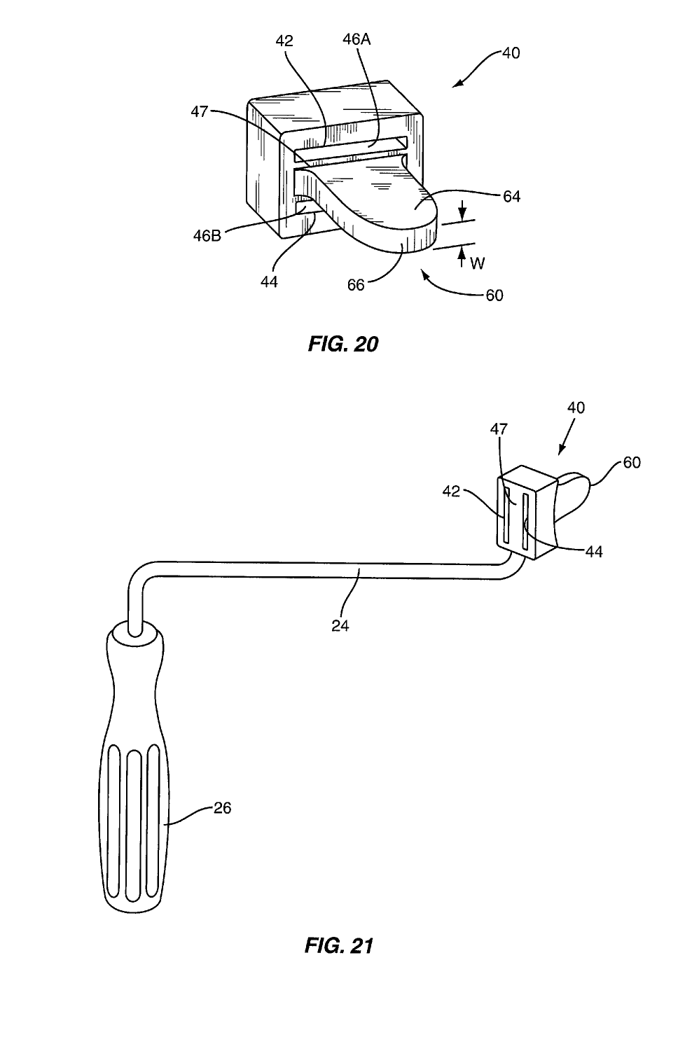

FIG. 20 illustrates an embodiment of a guide body 40 featuring a spacer 60 including a single prong 64 extending outward from a distal face of the guide body 40. The spacer 60 is positioned between the first and second edges 42, 44. Each of the first and second edges 42, 44 is respectively associated with an aperture 46A, 46B that extends through the guide body 40 and through which the blades 58 of a corresponding cutting tool 50 passes. An intermediate wall 47 is disposed between the apertures 46A, 46B. The prong 64 extends from the intermediate wall 47. The intermediate wall 47 may serve to limit the depth to which the blades 58 pass through the apertures 46A, 46B. The prong 64 has a smooth tapered edge that narrows to a rounded end 66. The rounded end 36 eases the insertion between the vertebral members. The spacer 60 is aligned parallel with the first and second edges 42, 44. The spacer 60 has a width W sized to space the articular processes IP, SP a distance apart to align the first edge 42 with a first of the articular processes IP, SP and the second edge 44 with the second of the articular processes IP, SP. In the illustrated embodiment, the spacer 60 includes a substantially constant thickness W. In other embodiments, the spacer 60 may be wedged, rounded, or chamfered resulting in an varying thickness W.

FIG. 21 illustrates a handle 26 and elongated arm 24 attached to the guide body 40. The handle 26 allows the surgeon to position and hold the guide body 40 relative to the articular processes IP, SP. The handle 26 allows the surgeon to use tactile senses to position the guide body 40. In one embodiment, handle 26 is off-center from the guide body 40 such that the surgeon can visually see the guide body 40 when holding the handle 26. The elongated arm 24 may include a variety of sizes and configurations. In one embodiment as illustrated in FIG. 21, a distal end of the elongated arm 24 attaches to a side wall of the guide body 40 to minimize interference with a cutting tool 50.

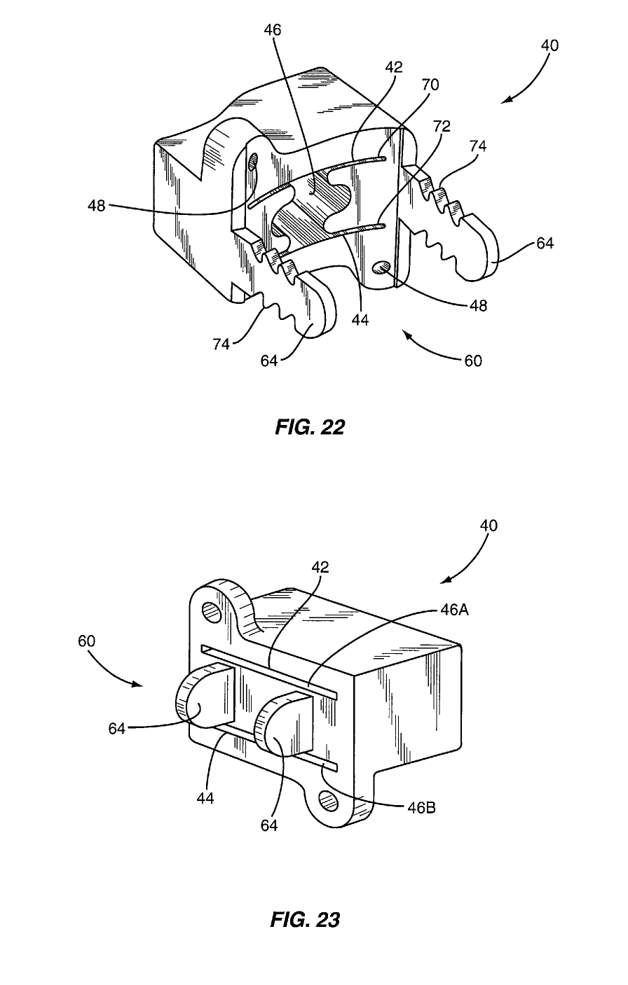

In the guide body 40 shown in FIGS. 20 and 21, the first and second edges 42, 44 are formed as part of separate apertures 46A, 46B. In another embodiment of a guide body 40 shown in FIG. 22, the first and second edges 42, 44 are formed as a portion of narrow slots 70, 72 that form a part of a contiguous aperture 46. In the illustrated embodiment, the guide body 40 includes a spacer 60 comprising two prongs 64 that extend outward from the guide body 40. Each prong 64 includes jagged edges 74 to reduce the likelihood of the guide body 40 inadvertently moving from between the articular processes IP, SP. In this embodiment, the jagged edges 74 are angled towards the guide body 40 such that insertion of the prongs 64 is not made more difficult or troublesome than with smooth edges (e.g., FIGS. 15, 16). The angled edges 74 catch on the articular processes IP, SP to prevent inadvertent removal.

FIG. 23 illustrates an embodiment of the guide 40 that includes first and second edges 42, 44 separated a distance apart by a solid guide body. The slots 46A, 46B including the first and second edges 42, 44 may include a variety of widths depending upon the application. The guide body 40 further includes a spacer 60 including a pair of prongs 64 that extend outward from a distal face of the guide body 40. The prongs 64 are spaced at points directly between the first and second edges 42, 44.

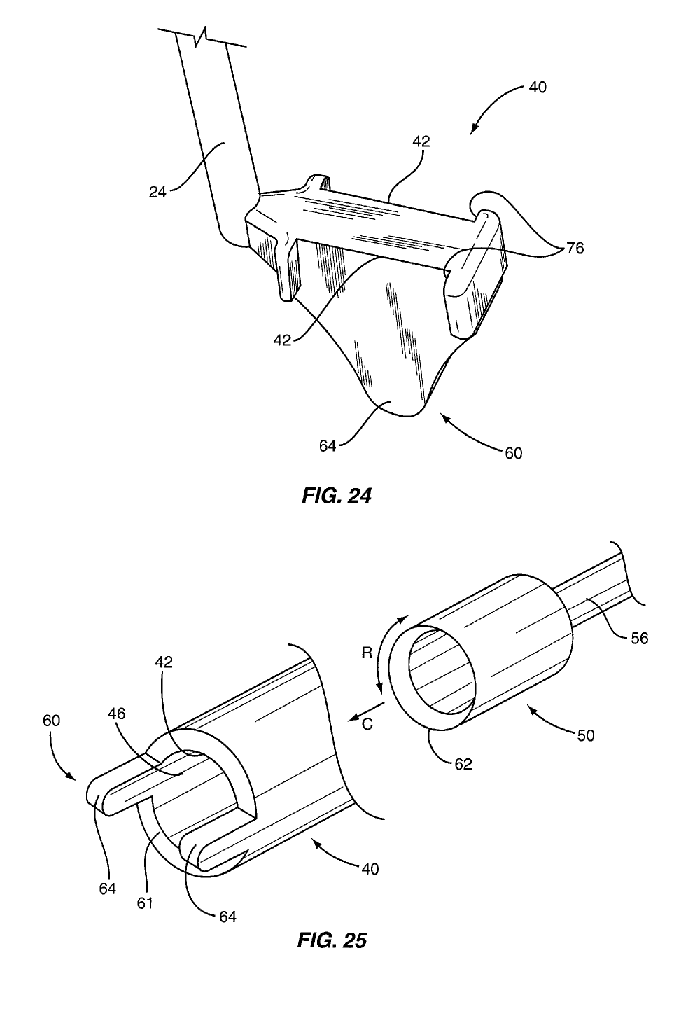

FIG. 24 illustrates another embodiment of the guide body 40 including exposed first and second edges 42, 44. The first and second edges 42, 44 are spaced a desired distance apart to support and guide the cutting tool 50. In one embodiment, shoulders 76 are positioned on one or both lateral ends of the edges 42, 44 to control the extent of blade 58 movement. Shoulders 76 prevent the blade 58 from inadvertently contacting sections of the articular processes IP, SP that are not to be contoured. In another embodiment, there are no shoulders 76. An elongated arm 24 may be attached to the guide body 40 to position the edges 42, 44. A spacer 60 comprised of one or more prongs 64 extends outward from one side of the guide body 40. The spacer 60 may include a variety of widths, including a first and second edge that align substantially with edges 42, 44. In another embodiment (not illustrated), there is no spacer 60.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. For instance, while the various Figures illustrate facet joint preparation for only one of the two facet joints, a similar configuration may exist at the facet joint located on the opposite lateral side of the spine. The descriptions disclosed herein are not intended to be limited to facet joints on a single side of the spine. Those skilled in the art will comprehend the symmetry and applicability of the various embodiments disclosed herein. The present embodiments are, therefore, to be considered in all respects as illustrative and not restrictive, and all changes coming within the meaning and equivalency range of the appended claims are intended to be embraced therein.

What is claimed is:

1. A device for contouring articular processes of a facet joint comprising:
a guide including a spacer extending outward in a longitudinal direction from a distal end of the guide and including a width to space apart the articular processes in a widthwise direction; and
a cutting member offset the spacer in the widthwise direction and including a cutting edge oriented to contour one of the articular processes in the longitudinal direction.

2. The device of claim 1 wherein the position of the cutting member and the guide are fixed with respect to each other.

3. The device of claim 2 wherein the cutting member is proximally disposed on one side of the guide.

4. The device of claim 2 wherein the cutting member and the guide are parallel to each other.

5. The device of claim 1 wherein the cutting member is moveable relative to the guide.

6. The device of claim 5 wherein the guide includes a guide surface and the cutting member moves in the longitudinal direction along the guide surface with the cutting edge contacting the one of the articular processes to contour the one of the articular processes.

7. The device of claim 5 wherein the guide includes a guide surface and the cutting member moves laterally transverse to the longitudinal direction along the guide surface with the cutting edge contacting the one of the articular processes to contour the one of the articular processes.

8. The device of claim 1 wherein the spacer comprises a plurality of prongs extending outward in the longitudinal direction from the distal end of the guide, the extensions including a substantially identical shape and size.

9. The device of claim 1 wherein the spacer comprises a single prong extending outward in the longitudinal direction from the distal end of the guide.

10. A device for contouring first and second articular processes of a facet joint comprising:
a guide including a spacer extending outward in a longitudinal direction from a distal end of the guide and including a width to space apart the first and second articular processes in a widthwise direction; and
a first cutting member offset a first side of the spacer in the widthwise direction and including a first cutting edge oriented to contour the first articular process in the longitudinal direction; and
a second cutting member offset a second side of the spacer in the widthwise direction and including a second cutting edge oriented to contour the second articular process in the longitudinal direction.

11. The device of claim 10 wherein the position of the first and second cutting members and the guide are fixed with respect to each other.

12. The device of claim 11 wherein the first and second cutting members are proximally disposed on opposite sides of the guide.

13. The device of claim 11 wherein the first and second cutting members and the guide are parallel to each other.

14. The device of claim 10 wherein the first and second cutting members are coupled to each other and moveable relative to the guide.

15. The device of claim 14 wherein the guide includes a first guide edge and a second guide edge, the first cutting member moves along the first guide surface with the first cutting edge contacting the first articular process and the second cutting member moves along the second guide edge with the second cutting edge contacting the second articular process.

16. The device of claim 15 wherein the first and second cutting members move along the first and second guide edges in the longitudinal direction.

17. The device of claim 15 wherein the first and second cutting members move along the first and second guide edges in a lateral direction.

18. The device of claim 15 wherein the spacer is disposed between the first and second guide edges.

19. The device of claim 15 wherein the first and second guide edges are formed by edges of a single aperture.

20. The device of claim 15 wherein the first and second guide edges are formed respectively by edges of a first and a second aperture.

21. The device of claim 10 wherein the spacer comprises a plurality of prongs extending outward in the longitudinal direction from the distal end of the guide, the extensions including a substantially identical shape and size.

22. The device of claim 10 wherein the spacer comprises a single prong extending outward in the longitudinal direction from the distal end of the guide.

23. A method of preparing articular processes for fusing a facet joint, the method comprising the steps of:
inserting a spacer between a first and a second articular process of the facet joint and spacing apart the first and second articular processes a predetermined width;
guiding a cutting edge relative to the spacer;
engaging the cutting edge with the first articular process at a location beyond the predetermined width; and
forcing the cutting edge into and contouring the first articular process while maintaining the cutting edge beyond the predetermined width.

24. The method of claim 23 wherein the step of forcing the cutting edge into and contouring the first articular process comprises inserting the spacer deeper into the facet joint.

25. The method of claim 23 further comprising securing the spacer to the first and second articular processes.

26. The method of claim 23 wherein the step of guiding a cutting edge relative to the spacer comprises inserting a cutting tool through a guide body on which the spacer is formed.

27. The method of claim 23 wherein the step of forcing the cutting edge into and contouring the first articular process comprises moving the cutting edge parallel to the spacer.

28. The method of claim 23 wherein the step of forcing the cutting edge into and contouring the first articular process comprises moving the cutting edge laterally back and forth relative to the spacer.

29. The method of claim 23 further comprising
guiding a second cutting edge relative to the spacer;
engaging the second cutting edge with the second articular process at a second location beyond the predetermined width and opposite the first cutting edge; and
forcing the second cutting edge into and contouring the second articular process while maintaining the second cutting edge beyond the predetermined width.

30. The method of claim 29 wherein the first and second cutting edges are formed on a single cutting tool.

31. The method of claim 29 wherein the first and second cutting edges are formed on separate cutting tools.

32. The method of claim 23 further comprising inserting an implant between the articular processes and causing the implant to osseointegrate with the facet joint.
